(19) United States
(12) Patent Application Publication
(10) Pub. No.: US 2006/0228250 A1
(43) Pub. Date: Oct. 12, 2006

(54) Method and composition for neutralizing odors

(76) Inventors: Robert C. Brown, Arlington, TX (US); Justin R. Brown, Arlington, TX (US)

Correspondence Address: CHALKER FLORES, LLP, 2711 LBJ FRWY, Suite 1036, DALLAS, TX, 75234, US

(21) Appl. No.: 11/082,318

(22) Filed: Mar. 17, 2005

(51) Int. Cl. A61L 9/00

(52) U.S. Cl. 422/5; 422/123; 424/76.1

(57) ABSTRACT

The present invention includes a composition, system and method for odor neutralization of an air circulation system or an enclosed area. The present invention includes a deodorizing composition for odor neutralization consisting essentially of from about 0.1% to about 2%, by weight of ethoxylated alkyphenol, from about 5% to about 20%, by weight of triethylene glycol, from about 25% to about 40%, by weight of ethanol, from about 1% to about 5%, by weight of ricinoleic acid, from about 3% to about 15%, by weight of water and citric acid.

### TECHNICAL FIELD OF THE INVENTION

The present invention relates generally to treating air circulation systems, and in particular, to odor neutralization of an air circulation system in an enclosed area.

### BACKGROUND OF THE INVENTION

Without limiting the scope of the invention, its background is described in connection with odor neutralization compositions, as an example.

The main purpose of an HVAC (e.g., heating, ventilating, and air conditioning) system is to provide the inside of an enclosure either a building or a vehicle with air that is clean and odor-free, and the temperature, humidity, and movement of the air are within certain comfort ranges so that it will be a comfortable and safe environment.

The main purpose of a ventilation system (e.g., heating, ventilating and air conditioning) is to provide the inside of an enclosure with comfortable, clean and odor-free air. Most ventilation systems share common components whether in a vehicle or a building. Commonly, ventilation systems include a duct system to transport the air to different locations, a fan to circulate the air, an air outlet and an inlet to bring fresh air into the enclosure or for recirculation of the air. Many ventilation systems include heating and cooling components such as heating coils, condensers and compressors. These heating and cooling components alter the humidity and temperature of the air being transported.

As the ventilation system operates, air and any contaminants suspended in the air are transported into the ventilation system. These contaminants (e.g., mold spores, microbes, bacteria, chemicals and dust) may be deposited on the surfaces within the ventilation system; and this accumulation may lead to many problems. Whether it is a vehicle or a building, the warm moisture humid environment of the ventilation system can result in bacterial or microbial growth and an unusual dank, musty odor emanating from the ventilation system.

One of the greatest annoyances to drivers and occupants of motor vehicles is the many objectionable odors associated with a vehicle. In addition to the above mentioned internal odor problems, the vehicle HVAC system has unique problems associated with the mobility of the vehicle. As the vehicle travels, it may encounter objectionable odors from a number of sources including environmental factors like noxious fumes from automobile exhaust and industrial effluent, as well as contaminants transported into the vehicle, e.g., debris, garbage, animals and the like.

Furthermore, the vehicle HVAC system is used to cool or heat the vehicle interior, often there is a problem in that the body sweat of the driver and passengers, spills, dog odors and cigarette smoke slowly accumulating in the cabin and/or ventilation ducts and the machinery and equipment in the HVAC system. Furthermore, the build-up of mold, bacteria and fungi inside the vehicle can lead to offensive and undesirable odors in automobile.

A variety of devices have been developed for covering or hiding odors emanating from an enclosure (e.g., buildings and vehicles) including the HVAC system, carpet, upholstery, plastics and other surfaces. For example, fragrance supplying devices for systems, which eliminate unpleasant odors and emit pleasant perfumes are commercially available, but it is difficult to maintain a pleasant atmosphere over a long period. Generally, fragrance supplying devices initially release large amounts of the deodorant or fragrance and gradually release less and less along with the passage of time. In addition, since the deodorant or fragrance is emitted continuously after the devices are opened, the driver and passengers eventually become physically “acclimated” to it and it loses effect. To solve these problems, Japanese Unexamined Utility Model Publication (Kokai) No. 1-75435, for example, proposes a fragrance supplying apparatus with a lid, which opens and closes to supply a fragrance in a vehicle intermittently.

A variety of devices has been developed for disinfecting and/or deodorizating a ventilation system of an enclosed system, (e.g., a building, a house, a room, an automobile), however, such known systems are expensive, generally difficult to manufacture and are generally intended for permanent use within the automobile or enclosure.

For example, U.S. Pat. No. 5,297,988 to Nishino, et al. disclose a fragrance supplying apparatus for a vehicle for supplying the interior of an air-conditioning duct of the vehicle with a fragrance or deodorant that is carried by a flow of air from the blower of the air-conditioning system into the cabin of the vehicle, the fragrance supplying apparatus including a fragrance supplying apparatus having control valves for controlling the supply of the fragrance or deodorant into the air-conditioning duct, a random number producing unit for producing random numbers and a control unit for controlling the control valves of the fragrance supplying unit based on the random numbers to set varying patterns of supply and cessation of the fragrance or deodorant.

Another example, U.S. Pat. No. 4,913,034 to Ripple, et al. discloses an air handling duct structure of an automotive vehicle having inlet and outlet ends and liquid heat exchange structure serially disposed within the duct structure intermediate the ends as well as blower structure serially disposed in the duct structure at a location therealong spaced intermediate the inlet end and the heat exchange structure includes liquid injection structure opening into an exposed wall portion of the duct structure at the aforementioned location thereof and is operative, when actuated, to inject a deodorizing liquid (and also possibly a disinfectant liquid) into the interior of the duct structure at the aforementioned location therealong.

Additionally, the interior of homes, cars, buildings and other enclosures can accumulate odors from various sources including body odors such as a sweat odor, hircismus odor, foot odor, scalp odor and life odors in the home including a tobacco odor and pet odor can create an inhospitable environment resulting in a desire for simple deodorization.

In addition to residential enclosures factories, clinics and shelter often accumulate odors from feces, urine, body odors, odors from bedding and stored chemicals, foods, and other materials can create an inhospitable environment resulting in need for a simple deodorization composition that will access hard to reach areas.

Many current deodorizing methods and compositions only mask the odor with a perfume and have an insufficient effect resulting in an increase in discomfort as a reverse effect.

The foregoing problems have been recognized for many years and while numerous solutions have been proposed, none of them adequately address all of the problems in a single device, e.g., inexpensive systems and efficient.

### SUMMARY OF THE INVENTION

The present inventors recognized a need for a deodorizing system that would accommodate various different areas (e.g., enclosures, rooms, HVAC systems) and configurations, while eliminating the foregoing problems. In accordance with the present invention, a method, system device and system are provided that include a deodorizing composition for odor neutralization that may be used in a variety of different forms, e.g., solids, liquids, vapors, solutions, aerosols, foggers, pump sprays, gels, dry formulation, additive, for other compositions and the like. The deodorizing composition allows effective directional delivery to an agent into a ventilation system, a surface, an enclosure or similar area. An object of the invention is to provide a deodorizing composition suitable for deodorizing spaces, surfaces and fiber products.

The deodorizing composition of the present invention has numerous uses and applications deodorizing vehicle malodors. The deodorizing composition may be used to neutralize the malodors in the interior of homes, cars, buildings and other enclosures that have accumulated odors from various sources including body odors such as a sweat odor, hircismus odor, foot odor, scalp odor and life odors in the home including tobacco odor and pet odor and created an inhospitable environment requiring deodorization. Furthermore, the present invention may be used to eliminate odors in industrial settings and enclosures like factories, clinics and shelter, which often accumulate odors from feces, urine, body odors, odors from bedding and storage areas (e.g., for chemicals, foods and paints), drycleaners, cleaners and school cafeterias. In addition, other materials can create an inhospitable environment resulting in a need for a simple deodorization composition that will access hard to reach areas.

The present invention may also be adapted for use in industrial applications where deodorizing and odor elimination are critical including car dealerships, car rental offices, carwashes, car cleaners, bus stations, schools, carpet cleaners, drycleaners, factories, farms, kennels, veterinary offices, garages, laundromats, repair shops, restaurants, gyms, spas, health clubs and the like.

Additionally, the present invention may be incorporated into a spray container that may be used in conjunction with a pivotable container holder, in instances where positioning containers and directing delivery of compounds is critical. For example, the pivotable container holder can be used to direct the delivery of a deodorization compound in a container to an area in need of deodorizing. The pivotable container holder can also be used to direct the delivery into an area or onto a surface or in a direction to maximize the distribution of the fragrance.

The enclosure to which the deodorizating compound may be delivered includes a sealed enclosure having one or more sidewalls, a top and a bottom or unsealed enclosure having one or more wall. Furthermore, the enclosure may also be an area, e.g., a lot of land, a yard, a pasture, a dock, a roof top or similar area. The enclosure may include a car, a house, a building, an office, a closet, a room, an attic, a crawl space, a cab, a tool box, a refrigerator, a freezer, a trailer, a trunk, a bus, a trolley, a mobile home, a bathroom, a bedroom, a garage, a warehouse, a storage unit, a clubhouse, a pantry, a laundry, a shed, a kitchen, a toy box, a train, an RV, an airplane, a helicopter, boat or a combination thereof.

For example, the present invention provides a method, system and device for deodorizing vehicle malodors by inserting a container having a deodorizing compound into a container holder having a container holding portion connected to one or more supports extending generally perpendicular from a generally planar base and one or more stops extending from the generally planar base, wherein the one or more stops restrict the movement of the container, adjusting the container to the desired position and activating the container, whereby the agent is dispersed. The position may be adjusted by placing the container holder on the floor, rotating the container in the container holder portion to provide the desired coverage and rotating the angle of the container holder portion relative to the base and securing the container holder portion with one or more stops.

The present invention includes a pivotable container holder having a generally planar base, one or more supports extending generally perpendicular from the generally planar base, a container holding portion having a container aperture for fitting frictionally, the container being attached pivotably to one or more of the one or more supports and one or more angled stops extending from the base, wherein the one or more stops restrict the movement of the container.

For example, the present invention includes from about 0.1% to about 2% by weight of ethoxylated alkyphenol, from about 5% to about 20% by weight of triethylene glycol, from about 25% to about 40% by weight of ethanol, from about 1% to about 5% by weight of ricinoleic acid, from about 3% to about 15% by weight of water and citric acid. The deodorizing composition may further include from about 0.1% to about 2% by weight of one or more perfumes and from about 30% to about 40% by weight of 1,1,1,2-tetrafluoroethane.

Another embodiment of the present invention includes a deodorizing system having spray for dispersing an odor neutralizing composition including a spray mode vessel containing a deodorizing composition consisting essentially of from about 0.1% to about 2% by weight of ethoxylated alkyphenol, from about 5% to about 20% by weight of triethylene glycol, from about 25% to about 40% by weight of ethanol, from about 1% to about 5% by weight of ricinoleic acid, from about 0.1% to about 10% by weight of citric acid, from about 0.1% to about 2% by weight of one or more perfumes and from about 30% to about 40% by weight of 1,1,1,2-tetrafluoroethane.

The present invention also includes an odor neutralizing composition atomizer including a housing having an inlet for connecting to a supply line, an outlet nozzle for emitting an atomized composition and a cavity being in flow communication with the inlet and a deodorizing composition consisting essentially of one or more surfactants, one or more solubilizing agents, one or more fatty acids, and one or more organic carboxylic acids.

In another example the present invention provide methods of deodorizing enclosures and surfaces and methods of treating undesirable odors in a ventilation system by placing a dispensing unit containing an odor neutralizing composition consisting essentially of one or more surfactants, one or more solubilizing agents, one or more fatty acids and one or more organic carboxylic acids into an enclosure, activating the dispensing unit, wherein the odor neutralizing composition is released into at least a portion of the enclosure, circulating the odor neutralizing composition throughout at least a portion of the enclosure.

The present invention also provides a method of neutralizing odors in an enclosure by including the steps of positioning a container comprising an odor neutralizing composition into an enclosure, wherein the odor neutralizing composition consists essentially of one or more surfactants, one or more solubilizing agents, one or more fatty acids and one or more organic carboxylic acids and contacting the enclosure with the odor neutralizing composition. The neutralizing composition may include ethoxylated alkyphenol, ethanol, water, triethylene glycol, ricinoleic acid, citric acid and 1,1,1,2-tetrafluoroethane.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

In accordance with the present invention, a method, system and composition are disclosed for use in reducing malodor in an enclosure. The present invention includes a method, system and composition that provided for the deodorizing a vehicle by inserting a container having the deodorizing composition into the vehicle either on a surface or in conjunction with a container holder. The container having the composition may be adjusted to deliver the deodorizing composition into the desired area by placing the container or the container holder on the floor, rotating the container to provide the desired coverage and adjusting the angle of the container. The container may then be activated to release the deodorizing composition into the vehicle.

The present invention also provides a method, system and composition for the deodorizing an enclosure by inserting a container having the deodorizing composition into the enclosure either on a surface or in conjunction with a container holder. The container may be adjusted to deliver the deodorizing composition into the desired area of the enclosure. The container may be positioned by placing the container or the container holder on the floor, rotating the container to provide the desired coverage and adjusting the angle of the container. The container may then be activated to release the deodorizing composition into the enclosure.

The container having the deodorizing composition may be hand-held, placed in a container holder or otherwise positioned to deliver the composition to the desired location. The container may be further adjusted to deliver the deodorizing composition into or onto the desired area. The container may be positioned by placing the container or the container holder on the floor, rotating the container to provide the desired coverage and adjusting the angle of the container. The container may then be activated to release the contents into the area.

The present invention is designed to be used in a variety of containers that deliver agents in a variety of different manners, including spraying, misting, fogging, smoking, atomizing, dispersing, evaporating, diffusing, coating, sputtering, propelling or combinations thereof. The agents may take any convenient form including liquids, solids, gases, foams, gels, or combinations thereof.

The contents of the container may be dispersed through a variety of different methods. The container may be an aerosol container, a spray container, a pump container, a container connected to an external pressure supply, a pressurized container, a container and fan combination or a combination thereof.

The container may be of different shapes and sizes. The most common container shape is a cylindrical, however, other shapes may be used such as spherical, conical, rectangular, polygonal or a combination thereof. The container may be an aerosol or pump container of the common size, e.g., 4, 6, 8, 10, 12 18, 20 and 24 ounces. Additionally, the container may be designed to fit relatively small containers with volumes equivalents measured in ounces to larger volumes containers with equivalent volumes measured in gallons.

For example, the composition of the present is designed to neutralization odors through the dispersion of the compositions throughout the enclosure so that the composition may contact the source of the odor. The method, device, composition and system of the present invention are designed to facilitate the delivery of the odor neutralizer into an enclosure or the duct system of a ventilation system; however, the present invention may be used to deliver the odor neutralizing composition onto and into a variety of surfaces and enclosures, e.g., a car, a house, a building, an office, a closet, a room, an attic, a crawl space, a cab, a tool box, a refrigerator, a freezer, a trailer, a trunk, a bus, a trolley, a mobile home, a bathroom, a bedroom, a garage, a warehouse, a storage unit, a clubhouse, a pantry, a laundry, a shed, a kitchen, a toy box, a train, an RV, an airplane, a helicopter, boat or a combination thereof. The present invention may be used in conjunction with a pivotable container holder to aid in the distribution of the composition to the desired location.

The composition of the present invention is designed to facilitate the delivery the odor neutralizing compound into the area adjacent to the container. The concentration of the individual components are combined to enhance particle size, particle size travel distance, foaming, content delivery and other factors relating to optimal delivery of the contents of the container.

The composition of the present invention is designed to permeate the area and allow the composition to access hard to reach areas as well as easily accessible areas. The composition may be directed in the air, onto a surface, into an enclosure, into a ventilation system or similar areas. Furthermore, the components of the composition may be adjusted or supplemented to change the characteristics of the delivery of the composition, e.g., larger particles, more water to increase evaporation times, more alcohol to increase solubility issues, and the like.

The method, device and system of the present invention are designed to facilitate the delivery of the contents of a container into the duct system of a ventilation system; however, the present invention may be used to deliver an agent into an enclosure. The enclosure may be a room, a part of a room, a wall, the space behind a wall, an attic or similar enclosure.

The enclosure may be a sealed enclosure having one or more sidewalls, a top and a bottom or unsealed enclosure having one or more wall. Furthermore, the enclosure may also be an area, e.g., a lot of land, a yard, a pasture, a dock, a roof top or similar area. The enclosure may include a car, a house, a building, an office, a closet, a room, an attic, a crawl space, a cab, a tool box, a refrigerator, a freezer, a trailer, a trunk, a bus, a trolley, a mobile home, a bathroom, a bedroom, a garage, a warehouse, a storage unit, a clubhouse, a pantry, a laundry, a shed, a kitchen, a toy box, a train, an RV, an airplane, a helicopter, boat or a combination thereof.

The present invention includes an odor neutralization composition for use in reducing malodor in an enclosure. The odor neutralization composition of the present invention may include an odor control agent having one or more fatty acids or fatty acid derivatives in a concentration between about 1 and 10% by weight. The fatty acids may be composed of different numbers of atoms and therefore differ in lengths, and/or different compositions, different constituting groups or of different classes. The fatty acids may be a saturated fatty acid having no double bonds, an unsaturated fatty acid has one double bond (e.g., olefinic) or a monounsaturated fatty acid contains one double bond.

Generally, fatty acids are named by the number of carbon atoms (n) and the number of double bonds (m) as (n:m). The nomenclature used herein for naming double bond position is to indicate the first double bond in the carbon backbone counting from the opposite end from the carboxyl group. The terminal carbon atom is called the omega carbon atom. Furthermore, the composition may be the salt form of the active fatty acid ingredient. The salt may be the salt selected from the group consisting of an alkali metal salt, an ammonium salt, a zinc salt, an aluminum salt and mixtures thereof.

One embodiment of the present invention includes the fatty acid known by the common name ricinoleic acid. Other embodiments may use different fatty acids including cis-4-decenoic, cis-9-decenoic, cis-5-lauroleic, cis-4-dodecenoic, cis-9-tetradecenoic, cis-5-tetradecenoic, cis-4-tetradecenoic, cis-9-hexadecenoic, cis-6-octadecenoic, cis-9-octadecenoic, cis-11-octadecenoic, cis-9-eicosenoic, cis-11-eicosenoic, cis-11-docosenoic, cis-13-docosenoic, cis-15-tetracosenoic, 12-hydroxy-(cis)-9-octadecenoic acid, (z)-9-tetradecenoic acid, (z)-9-hexadecenoic acid, (e)-9-hexadecenoic acid, (z)-11-octadecenoic acid, (z)-9-octadecenoic acid, (e)-9-octadecenoic acid, (e,e)-9,12-octadecadienoic acid, (z,z)-9,12-octadecadienoic acid, (e,e)-9,11-octadecadienoic acid, (z,z,z)-9,12,15-octadecatrienoic acid, (z,z,z)-6,9,12-octadecatrienoic acid, (z,z,z,z)-6,9,12,15-octadecatetraenoic acid, (z)-11-ecosenoic acid, (z,z,)-11,14-ecosadienoic acid, (z,z,z)-5,8,11-eicosatrienoic acid, (z,z,z)-11,14,17-eicosatrienoic acid, (z,z,z)-8,11,14-eicosatrienoic acid, (z)-9-octadecenoic acid methyl ester, trans-9-octadecenoic acid methyl ester or a combination thereof.

Still other embodiments of the present invention may use fatty acids including obtusilic acid, caproleic acid, lauroleic acid, linderic acid, myristoleic acid, physeteric acid, tsuzuic acid, palmitoleic acid, petroselinic acid, oleic acid, vaccenic (asclepic) acid, gadoleic acid, gondoic acid, cetoleic acid, erucic acid, nervonic acid, olefinic acid, palmitelaidic acid, vaccenic acid, elaidic acid, linoelaidicic acid, linoleic acid, isolinoleic acid, gamma-linolenic acid, moroctic acid, eicosenoic acid, eicosadienoic acid, mead acid, eicosatrienoic acid, dihomo-g-linolenic acid, ricinoleic acid or a combination thereof.

Still other embodiments may include fatty acids that have one or more modification including the addition of groups to the fatty acid chain, e.g., methyl, methoxy, methyl ester, hydroxyl, carboxyl, addition of ring structures. The modification may occur at various positions in the carbon chain, which can be saturated, unsaturated or monoenoic. Furthermore, the fatty acid may have more than one modification per fatty acid chain and the modifications may be similar (e.g., polyhydroxy fatty acids) or different.

The fatty acid chain may be derivatives or analogues of fatty acids and be substituted by one or more atoms. For example, the fatty acids may include a sulfur-substituted fatty acid analogue or nitrogen derivatives of fatty acids, e.g., amino acids, hydrazides, acid azides, nitriles, isocyanates, amines or a combination thereof.

Additionally, fatty acids of different types may be used including cis-monoenoic fatty acids, trans-monoenoic fatty acids, monoenoic acids, epoxy acids, acetylenic fatty acids, polyunsaturated acetylenic fatty acids, fatty acid amides, methoxy fatty acids, keto fatty acids or a combination thereof. For example, the present invention may contain one or more trans-monoenoic fatty acids, cis-monoenoic fatty acids, monoenoic acids, trans fatty acid, acetylenic fatty acids, polyunsaturated acetylenic fatty acids, epoxy acids, 1 cyclopropane acids, cyclopropene acids, cyclohexyl acids, cyclopentenyl acids and cyclohexenyl acids, hydroxylated fatty acids or a combination thereof.

In one embodiment, the present invention may contain one or more t9-octadecenoic acids, t11-octadecenoic acids, 9,10-epoxystearic acids and 9,10-epoxyoctadec-12-enoic acids, 12,13-epoxy-9-octadecenoic acids, 2-hydroxy acids, cerebronic acids, hydroxynervonic acids, 3-hydroxy acids (or β-hydroxy acids), 10-hydroxy acids, polyhydroxy fatty acids, 2-hydroxytetracosanoic acids, 2-hydroxy-15-tetracosenoic acids, trans 10-hydroxydec-2-enoic acids, 10-hydroxydecanoic acids, 9-hydroxydec-2-enoic acids, 14-hydroxy-11,17-eicosadienoic acids, 12-hydroxy-9,15-octadecadienoic acids, 14-hydroxy-11-eicosenoic acids, 12-hydroxy-9-octadecenoic acids, amide of 9-octadecenoic acids, 13-cis-docosenoic acids, 2-methoxy-5-hexadecenoic acids, 2-methoxy-6-hexadecenoic acids, 2-methoxy hexadecanoic acids, 7-methoxy-4-tetradecenoic acids, 2-methoxy-13-methyltetradecanoic acids, 2-methoxylated C15-C16 fatty acids, tr9-keto-2-decenoic acids, 6,9-octadecenynoic acids, 9-octadecynoic acids, 6-octadecynoic acids, 6-octadecen-9-ynoic acids, t10-heptadecen-8-ynoic acids, t11-octadecen-9-ynoic acids, 9-octadecen-12-ynoic acids or a combination thereof.

In other embodiments, the present invention may contain auricolic acid, ricinoleic acid, densipolic acid, oleamide, erucamide, dodeca thia acetic acid, tetradeca thia acetic acid, tariric acid, stearolic acid santalbic acid, pyrulic acid, crepenynic acid, coronaric acid, t3-hexadecenoic acid, elaidic acid, undecylenic acid, t-vaccenic acid or a combination thereof. In addition the fatty acids of the present invention may include modifications including methyl substituted, ethyl substituted, hydroxyalkyl substituted, branched, cationic, quaternary ammonium, anionic, amphoteric and mixtures thereof. The present invention may also include one or more surfactants, emulsifying agents, dispersants, spreading agents, wetting agents or penetrants. Conventional surfactants such as anionic surfactants and nonionic surfactants can be used.

Surfactants may be used anywhere there is a need for increased surface activity and provide excellent all-purpose detergency and wetting, as well as solubilization and emulsification. The present invention may include one or more nonionic surfactant, e.g., tergitol, poly(oxy-1,2-ethanediyl) α-(4-nonylphenyl)-.omega.-hydroxy, ethoxylated alkyphenol, alkoxylated linear alcohols, ethoxylated linear alcohols, glycols, polyethylene, mono(p-nonylphenyl)ether, polyoxyethylene alkyl ethers and polyoxyethylene alkyl arylether, polyoxyethylene arylether, esters of fatty acids with polyhydric alcohols, nonoxynol-9 or a combination thereof.

The present invention may also include nonionic surfactants, e.g., polyoxyethylene alkyl ethers, polyoxyalkylene alkyl ethers, polyoxyalkylene alkyl ethers, polyoxyethylene derivatives, xyethylene oxypropylene block copolymers, sorbitan fatty acid esters, polioxyethylene sorbitan fatty acid esters, polioxyethylene sorbitol fatty acid esters, glycerol fatty acid esters(reodol ms seriese), glycerol fatty acid esters(excel seriese), polyoxyethylene fatty acid esters, polyoxyethylene alkyl amines, alkylol amides, fatty acid polyhydric alcohol polyoxyethylenes, fatty acid esters of sucrose, block copolymers of ethylene oxide, propylene oxide and combinations thereof.

Other nonionic surfactant may include fatty alcohols having the general structure ROH, fatty acids having the general structure having the general structure R(CO)OH, fatty esters having the general structure R(CO)OR′, glycerol esters having the general structure H(CHOZ)(CHOZ′)(CHOZ″)H, glycol esters having the general structure R(CHOZ)(CHOZ′)H, alkyl phenols having the general structure R—PH—O—PEO—H, acetylenic glycols having the general structure R[(COH)CH₃CH₃CH3]C^{o}C[(COH)CH₃]R, alkanolamines, alkanolamides, polyethoxylated mercaptans RS—PEO—H, sorbitol, sorbitan derivatives and combinations thereof. In addition to nonionic surfactants, other surfactants may also be used for example ethoxylated and propoxylated surfactants, fluorinated surfactants, siloxane surfactants and combinations thereof.

Anionic surfactant may also be used and include metallic soaps, salts of sulfuric esters such as sodium alkylsulfate, alkylbenzene sulfonates such as sodium alkylbenzene sulfonate, alkylnaphthalenesulfonates such as sodium alkylnaphthalene sulfonate, salts of dialkyl 2-sulfosuccinates such as sodium dialkyl 2-sulfosuccinate, polycarboxylic acid-based surfactants, α-olefin sulfonates, polyoxyethylenedistyrenephenylether sulfate ammonium salt, sodium ligninsulfonate and potassium ligninsulfonate.

Other embodiments may use surfactants that are cationic surfactants (e.g., alkylamines having the general structure R(NR′)R″, quaternary amines having the general structure R[(N+R′)R″]R′″X, imidazolines, dialkylamine oxides having the general structure R—[(NO)R′]R″ or combinations thereof), anionics, carboxylates, salts of fatty acids having the general structure R(CO)O— M+, salts of multiple acids having the general structure HO(CO)(CHOH)(CO)O— M+, sarcosine derivatives having the general structure R(CO)(NCH₃)CH₂(CO)O—NA+, salts of tall oil acids and combinations thereof.

Other surfactants that may be used with the present invention may include sulfonates including sodium alkyl sulfonates having the general structure RSO₃—NA+, alpha-olefin sulfonates, linear alkylbenzene sulfonates having the general structure R(CHR′)—PH—SO₃—NA+, other aromatic sulfonates including isethionates having the general structure R(CO)OCH₂CH₂SO₃— M+, sulfosuccinates having the general structure RO(CO)CH₂CH(SO₃— M+)(CO), taurates having the general structure R(CO)(NR′)CH₂CH₂SO₃— M+, alcohol sulfates having the general structure having the general structure R—O—SO₃— M+, alkyl phenol sulfates having the general structure R—PH—O—PEO—SO₃— M+, sulfated triglycerides and combinations thereof.

Additionally, the present invention may include phosphates including alcohol phosphates having the general structure RO[(PO)OZ]O— M+, inorganic phosphates having the general structure M′+[O—(PO)O— M+]NO— M″+, zwitterionics (amphotherics) including amino acids having the general structure R—(N+H₂)CH₂CH₂(CO)O, imino acids having the general structure R—[(N+H)CH₂CH₂(CO)OH]—CH₂CH₂(CO)O, betaines having the general structure R[N+(CH₃)₂](CH₂)X(CO)O, imidazolines and phospholipids having the general structure R(CO)OCH₂[CHO(COR′]CH₂O(PO₂—)X+.

The present invention may also contain one or more perfumes in a concentration range of between about 0.1 to 1.0% to 1.0 to 10% by volume. The final volume of perfumes used may vary depending on the application and the specific perfume selected. The perfume may be selected from the group consisting of ambrox, bacdanol, benzyl salicylate, butyl anthranilate, cetalox, damascenone, alpha-damascone, gamma-dodecalactone, ebanol, herbavert, cis-3-hexenyl salicylate, alpha-ionone, beta-ionone, alpha-isomethylionone, lilial, methyl nonyl ketone, gamma-undecalactone, undecylenic aldehyde, and mixtures thereof. The present invention may also include a perfume selected from the group consisting of allyl amyl glycolate, allyl caproate, amyl acetate, amyl propionate, anisic aldehyde, anisyl acetate, anisole, benzaldehyde, benzyl acetate, benzyl acetone, benzyl alcohol, benzyl formate, benzyl iso valerate, benzyl propionate, beta gamma hexenol, calone, camphor gum, laevo-carveol, d-carvone, laevo-carvone, cinnamic alcohol, cinnamyl acetate, cinnamic alcohol, cinnamyl formate, cinnamyl propionate, cis-jasmone, cis-3-hexenyl acetate, coumarin, cuminic alcohol, cuminic aldehyde, Cyclal C, cyclogalbanate, dihydroeuginol, dihydro isojasmonate, dimethyl benzyl carbinol, dimethyl benzyl carbinyl acetate, ethyl acetate, ethyl aceto acetate, ethyl amyl ketone, ethyl anthranilate, ethyl benzoate, ethyl butyrate, ethyl cinnamate, ethyl hexyl ketone, ethyl maltol, ethyl-2-methyl butyrate, ethyl methylphenyl glycidate, ethyl phenyl acetate, ethyl salicylate, ethyl vanillin, eucalyptol, eugenol, eugenyl acetate, eugenyl formate, eugenyl methyl ether, fenchyl alcohol, flat acetate(tricyclo decenyl acetate), fructone, frutene(tricyclo decenyl propionate), geraniol, geranyl oxyacetaldehyde, heliotropin, hexenol, hexenyl acetate, hexyl acetate, hexyl formate, hinokitiol, hydratropic alcohol, hydroxycitronellal, hydroxycitronellal diethyl acetal, hydroxycitronellol, indole, isoamyl alcohol, iso cyclo citral, isoeugenol, isoeugenyl acetate, isomenthone, isopulegyl acetate, isoquinoline, keone, ligustral, linalool, linalool oxide, linalyl formate, lyral, menthone, methyl acetophenone, methyl amyl ketone, methyl anthranilate, methyl benzoate, methyl benzyl acetate, methyl cinnamate, methyl dihydrojasmonate, methyl eugenol, methyl heptenone, methyl heptine carbonate, methyl heptyl ketone, methyl hexyl ketone, methyl isobutenyl tetrahydropyran, methyl-N-methyl anthranilate, methyl beta naphthyl ketone, methyl phenyl carbinyl acetate, methyl salicylate, nerol, nonalactone, octalactone, octyl alcohol (octanol-2), para-anisic aldehyde, para-cresol, para-cresyl methyl ether, para hydroxy phenyl butanone, para-methoxy acetophenone, para-methyl acetophenone, phenoxy ethanol, phenoxyethyl propionate, phenyl acetaldehyde, phenylacetaldehyde diethyl ether, phenylethyl oxyacetaldehyde, phenyl ethyl acetate, phenyl ethyl alcohol, phenyl ethyl dimethyl carbinol, prenyl acetate, propyl butyrate, pulegone, rose oxide, safrole, terpineol, vanillin, viridine, allyl heptoate, amyl benzoate, anethole, benzophenone, carvacrol, citral, citronellol, citronellyl nitrile, cyclohexyl ethyl acetate, cymal, 4-decenal, dihydro isojasmonate, dihydro myrcenol, ethyl methyl phenyl glycidate, fenchyl acetate, florhydral, gamma-nonalactone, geranyl formate, geranyl nitrile, hexenyl isobutyrate, alpha-ionone, isobornyl acetate, isobutyl benzoate, isononyl alcohol, isomenthol, para-isopropyl phenylacetaldehyde, isopulegol, linalyl acetate, 2-methoxy naphthalene, menthyl acetate, methyl chavicol, musk ketone, beta naphthol methyl ether, neral, nonyl aldehyde, phenyl heptanol, phenyl hexanol, terpinyl acetate, Veratrol, yara-yara, and mixtures thereof.

Some embodiments of the present invention may also include one or more emollients. Although any compatible emollient may be used, common emollients include petrolatum, mineral oil, micro-crystalline wax, polyalkene, paraffin, cerasin, ozokerite, polyethylene and perhydrosqualene, dimethicones, alkyl siloxane, polymethylsiloxane, methylphenylpolysiloxane, and mixtures thereof.

The present invention may also include organic carboxylic acids, e.g., 2-hydroxy-1,2,3-propanetricarboxylic acid, citric acid. In some instances the organic carboxylic acids may be used to stabilize the composition, act as an antioxidant in fats and oils, protect the composition against oxidation, stabilize the pH of the composition, adjust the pH of the composition or a combination thereof.

The present invention also includes one or more alcohols. The alcohol may be used to reduce the risk of material aggregating undesirably and enhances the dispersement of the compound. The alcohol may be any compatible alcohol known to persons of skill in the art. Common examples of alcohols used in the present invention include methanol, ethanol, propanol, isopropanol and the like.

Additionally, some nonlimiting examples include methanol, ethanol, isopropanol, hexanol, 1,2-hexanediol, hexylene glycol, (e.g. 2-methyl-2,4-pentanediol), isopropylen glycol(3-methyl-1,3-butanediol), 1,2-butylene glycol, 2,3-butylene glycol, 1,3-butylene glycol, 1,4-butylene glycol, 1,3-propylene glycol, 1,2-propylene glycol, isomers of cyclohexanedimethanol, isomers of propanediol, isomers of butanediol, the isomers of trimethylpentanediol, the isomers of ethylmethylpentanediol, alcohol ethoxylates of 2-ethyl-1,3-hexanediol, 2,2,4-trimethyl-1,3-pentanediol, alcohol ethoxylates of 2,2,4-trimethyl-1,3-pentanediol glycerol, ethylene glycol, diethylene glycol, dipropylene glycol, sorbitol, 3-methyl-3-methoxybutanol, 3-methoxybutanol, 1-ethoxy-2-propanol, diethylene glycol monoethyl ether, diethylene glycol monopropyl ether, diethylene glycol monobutyl ether, triethylene glycol, monoethyl ether, erythritol, and mixtures of solvents and plasticizers.

The present invention also includes triethylene glycol. The triethylene glycol may be in concentrations between 10 and 25% by weight. In addition, other glycols and similar compounds may be used in the present invention including alkanolamides, silicone glycols, alkyl benzene sulfonates, alcohol ethoxylated, phosphate esters, alkylphenol ether sulfates, alkylaryl sulfonates, nonylphenoxypoly-(ethyleneoxy)ethanol, polyethylene glycol, monostearate, sulfonates, siloxanes, polyglycols, polyvinyl alcohol, polyester glutarate, caprate/caprylate, long chain alkyl ether, dialkyl diester, glutarate, and combinations thereof.

The present invention may also include water in concentrations between 3 and 15% by weight. Optionally, in addition to water, the carrier can further include solvents and/or plasticizers. Acceptable solvents and plasticizers include compounds having from one to ten carbons. The following non-limiting classes of compounds are suitable: mono-alcohols, diols, polyhydric alcohols, ethers, ketones, esters, organic acids, and alkyl glyceryl ethers, and hydrocarbons.

Additionally, the present invention may include exemplified water; alcohols such as methanol, ethanol, n-propanol, i-propanol, n-butanol, i-butanol, sec-butanol, t-butanol, n-octanol, and cyclohexanol; ethers such as ethyl ether, propyl ether, isopropyl ether, dimethoxyethane, cyclic ethers (e.g., dioxane, tetrahydrofuran), mono- or di-alkylene glycol monoalkylethers (e.g., ethylene glycol monomethylether, ethylene glycol monoethylether, diethylene glycol monoethylether); ketones such as acetone, methyl ethyl ketone, methyl isobutyl ketone, and cyclohexanone; esters such as ethyl acetate and butyl acetate; halogenated hydrocarbons such as carbon tetrachloride, chloroform, dichloromethane and 1,2-dichloroethane; aliphatic hydrocarbons such as hexane and octane; alicyclic hydrocarbons such as cyclohexane and cycloheptane; aromatic hydrocarbons such as benzene, toluene and xylene; nitrites such as acetonitrile; carboxylic acids such as formic acid and acetic acid; and aprotic polar solvents such as N,N-dimethylformamide, dimethyl sulfoxide, and pyridine. These solvents can be used either singly or in combination.

The present invention may also include a dispenser in the form of an aerosol spray presentation from a pressurized container, a pump, a spray, a nebuliser or other pressurized delivery systems. In addition, the composition may be a solid, effervescent compound. The deodorizing composition of the present invention has numerous uses and applications deodorizing vehicle malodors. The deodorizing composition may be used to neutralize the malodors in the interior of homes, cars, buildings and other enclosures that have accumulated odors from various sources including body odors such as a sweat odor, hircismus odor, foot odor, scalp odor and life odors in the home including tobacco odors and pet odors that create an inhospitable environment requiring deodorization.

Furthermore, the present invention may be used to eliminate odors in industrial settings and enclosures like factories, clinics and shelter often accumulate odors from feces, urine, body odors, odors from bedding and stored chemicals, foods, paints, cleaners, drycleaners, school cafeterias and other materials can create an inhospitable environment resulting in need for a simple deodorization composition that will access hard to reach areas. Furthermore, the present invention may be adapted for delivery for industrial applications including car dealerships, car rental offices, carwashes, car cleaners, bus stations, schools, carpet cleaners, drycleaners, factories, farms, kennels, veterinary offices, garages, laundromats, repair shops, restaurants, gyms, spas, health clubs and the like.

In addition to pressurized delivery systems, the composition of the present invention may be conveniently delivered in the form of a dry powder dispersion. The present invention may use dimethyl ether, chlorofluorocarbons, non-soluble compressed gasses (e.g. compressed air and nitrogen), soluble compressed gasses (e.g. carbon dioxide), liquefied petroleum gas (LPG) aerosol propellant grade LPG containing high purity hydrocarbons and include a mixture of propane, isobutane and n-butane or a combination thereof as propellants.

Additionally, propellants for use in the present invention include propane, isobutane, n-butane, dimethyl ether, 1,1-difluoroethane, 1,1,1,2-tetrafluoroethane and mixtures thereof. In one embodiment, the propellant includes 1,1,1,2-tetrafluoroethane. In another embodiment, the propellant is a mixture of propane and isobutane. The propellant may also be selected from chlorofluorocarbons, hydrofluorocarbons and from mixtures thereof.

The pressurized container, pump, spray or nebuliser embodiments may contain a solution or suspension of the composition, e.g. using a mixture of ethanol and the propellant as the solvent, which may additionally contain a lubricant, e.g. sorbitan trioleate.

The composition may also be formulated for delivery via an atomiser. Formulations for atomiser devices may contain solubilisers, emulsifiers or suspending agents, water, ethanol, glycerol, triethylene glycol, low molecular weight polyethylene glycols, fluorocarbons, nonionic surfactants, fatty acids. One embodiment of the present invention includes formulations for atomiser devices including the zinc salt of ricinoleic acid, ethoxylated alkyphenol, triethylene glycol, water, ethanol, citric acid and a suitable fragrance.

For example one embodiment of the present invention also includes a pressurized dilevery system including a spray can of deoderizer prepared as an active dispenser, incorporating the zinc salt of ricinoleic acid about 3%, ethoxylated alkyphenol about 1%, triethylene glycol about 18%, water about 7%, ethanol about 34%, citric acid, a suitable fragrance about 1% and 1,1,1,2-tetrafluoroethane about 35%. A person of ordinary skill in the art will recognize that the ethoxylated alkyphenol may be from about 0.1% to about 2% by weight; triethylene glycol may be from about 5% to about 20% by weight; ethanol may be from about 25% to about 40% by weight; ricinoleic acid may be from about 1% to about 5% by weight; water may be from about 3% to about 15% by weight, and citric acid.

The present invention may be used in conjunction with a device that supplies a stable base and positions the container containing the odor neutralizing composition to facilitate the distribution in a directional manner. The pivotable container holder may be used to facilitate the delivery of the contents of a container into the ducts of a ventilation system; however, the pivotable container holder may also be used to deliver an agent into an enclosure or onto a surface. The enclosure may be a room, a part of a room, a wall, the space behind a wall, an attic or similar enclosure. The pivotable container holder allows adjustment of the angle the container is held and the position of the delivery mechanism, e.g., nozzle, tube or tip.

The pivotable container holder includes a generally planar base, one or more supports extending generally perpendicular from the base, a container holding portion having a container aperture for fitting frictionally a container attached pivotably to one or more of the one or more supports and one or more stops extending from the base, wherein the one or more stops restrict the movement of the container. This configuration allows the container to be positioned at the desired angle to accommodate dispersion of the deodorization composition into the enclosure and induction into a ventilation system while increasing stability using a base and reducing the damage to the surrounding enclosure.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A deodorant composition for odor neutralization consisting essentially of:
from about 0.1% to about 2% by weight of ethoxylated alkyphenol;
from about 5% to about 20% by weight of triethylene glycol;
from about 25% to about 40% by weight of ethanol;
from about 1% to about 5% by weight of ricinoleic acid;
from about 3% to about 15% by weight of water; and
citric acid.

2. The deodorant composition of claim 1, further comprising from about 0.1% to about 2%, by weight of one or more perfumes.

3. The deodorant composition of claim 1, further comprising from about 30% to about 40%, by weight of 1,1,1,2-tetrafluoroethane.

4. The deodorant composition of claim 1, wherein the deodorant composition is contained within a spray vessel.

5. A odor neutralizing composition comprising:
one or more surfactants;
one or more solubilizing agents;
one or more fatty acids; and
one or more organic carboxylic acids.

6. The odor neutralizing composition of claim 5, further comprising one or more perfumes.

7. The odor neutralizing composition of claim 6, wherein the one or more perfumes are selected from the group of allyl amyl glycolate, allyl caproate, amyl acetate, amyl propionate, anisic aldehyde, anisyl acetate, anisole, benzaldehyde, benzyl acetate, benzyl acetone, benzyl alcohol, benzyl formate, benzyl iso valerate, benzyl propionate, beta gamma hexenol, calone, camphor gum, laevo-carveol, d-carvone, laevo-carvone, cinnamic alcohol, cinnamyl acetate, cinnamic alcohol, cinnamyl formate, cinnamyl propionate, cis-jasmone, cis-3-hexenyl acetate, coumarin, cuminic alcohol, cuminic aldehyde, Cyclal C, cyclogalbanate, dihydroeuginol, dihydro isojasmonate, dimethyl benzyl carbinol, dimethyl benzyl carbinyl acetate, ethyl acetate, ethyl aceto acetate, ethyl amyl ketone, ethyl anthranilate, ethyl benzoate, ethyl butyrate, ethyl cinnamate, ethyl hexyl ketone, ethyl maltol, ethyl-2-methyl butyrate, ethyl methylphenyl glycidate, ethyl phenyl acetate, ethyl salicylate, ethyl vanillin, eucalyptol, eugenol, eugenyl acetate, eugenyl formate, eugenyl methyl ether, fenchyl alcohol, flat acetate(tricyclo decenyl acetate), fructone, frutene(tricyclo decenyl propionate), geraniol, geranyl oxyacetaldehyde, heliotropin, hexenol, hexenyl acetate, hexyl acetate, hexyl formate, hinokitiol, hydratropic alcohol, hydroxycitronellal, hydroxycitronellal diethyl acetal, hydroxycitronellol, indole, isoamyl alcohol, iso cyclo citral, isoeugenol, isoeugenyl acetate, isomenthone, isopulegyl acetate, isoquinoline, keone, ligustral, linalool, linalool oxide, linalyl formate, lyral, menthone, methyl acetophenone, methyl amyl ketone, methyl anthranilate, methyl benzoate, methyl benzyl acetate, methyl cinnamate, methyl dihydrojasmonate, methyl eugenol, methyl heptenone, methyl heptine carbonate, methyl heptyl ketone, methyl hexyl ketone, methyl isobutenyl tetrahydropyran, methyl-N-methyl anthranilate, methyl beta naphthyl ketone, methyl phenyl carbinyl acetate, methyl salicylate, nerol, nonalactone, octalactone, octyl alcohol (octanol-2), para-anisic aldehyde, para-cresol, para-cresyl methyl ether, para hydroxy phenyl butanone, para-methoxy acetophenone, para-methyl acetophenone, phenoxy ethanol, phenoxyethyl propionate, phenyl acetaldehyde, phenylacetaldehyde diethyl ether, phenylethyl oxyacetaldehyde, phenyl ethyl acetate, phenyl ethyl alcohol, phenyl ethyl dimethyl carbinol, prenyl acetate, propyl butyrate, pulegone, rose oxide, safrole, terpineol, vanillin, viridine, allyl heptoate, amyl benzoate, anethole, benzophenone, carvacrol, citral, citronellol, citronellyl nitrile, cyclohexyl ethyl acetate, cymal, 4-decenal, dihydro isojasmonate, dihydro myrcenol, ethyl methyl phenyl glycidate, fenchyl acetate, florhydral, gamma-nonalactone, geranyl formate, geranyl nitrile, hexenyl isobutyrate, alpha-ionone, isobornyl acetate, isobutyl benzoate, isononyl alcohol, isomenthol, para-isopropyl phenylacetaldehyde, isopulegol, linalyl acetate, 2-methoxy naphthalene, menthyl acetate, methyl chavicol, musk ketone, beta naphthol methyl ether, neral, nonyl aldehyde, phenyl heptanol, phenyl hexanol, terpinyl acetate, Veratrol, yara-yara, and mixtures thereof.

8. The odor neutralizing composition of claim 5, further comprising one or more propellants.

9. The odor neutralizing composition of claim 8, wherein the one or more propellants comprise propane, isobutane, n-butane, dimethyl ether, 1,1-difluoroethane, 1,1,1,2-tetrafluoroethane, chlorofluorocarbons, hydrofluorocarbons, 1,1,1,2-tetrafluoroethane and combinations thereof.

10. The odor neutralizing composition of claim 5, further comprising a fragrant agent, a biocide agent, a biostatic agent or a combination thereof.

11. The odor neutralizing composition of claim 5, wherein one or more of the one or more fatty acids are in the form of an alkali metal salt, an ammonium salt, a zinc salt, an aluminum salt and mixtures thereof.

12. The odor neutralizing composition of claim 5, wherein the one or more fatty acids comprise ricinoleic acid, cis-4-decenoic, cis-9-decenoic acid, cis-5-lauroleic acid, cis-4-dodecenoic acid, cis-9-tetradecenoic acid, cis-5-tetradecenoic acid, cis-4-tetradecenoic acid, cis-9-hexadecenoic acid, cis-6-octadecenoic acid, cis-9-octadecenoic acid, cis-11-octadecenoic acid, cis-9-eicosenoic acid, cis-11-eicosenoic acid, cis-11-docosenoic acid, cis-13-docosenoic acid, cis-15-tetracosenoic acid, 12-hydroxy-(cis)-9-octadecenoic acid, (z)-9-tetradecenoic acid, (z)-9-hexadecenoic acid, (e)-9-hexadecenoic acid, (z)-11-octadecenoic acid, (z)-9-octadecenoic acid, (e)-9-octadecenoic acid, (e,e)-9,12-octadecadienoic acid, (z,z)-9,12-octadecadienoic acid, (e,e)-9,11-octadecadienoic acid, (z,z,z)-9,12,15-octadecatrienoic acid, (z,z,z)-6,9,12-octadecatrienoic acid, (z,z,z,z)-6,9,12,15-octadecatetraenoic acid, (z)-11-ecosenoic acid, (z,z,)-11,14-ecosadienoic acid, (z,z,z)-5,8,11-eicosatrienoic acid, (z,z,z)-11,14,17-eicosatrienoic acid, (z,z,z)-8,11,14-eicosatrienoic acid, (z)-9-octadecenoic acid methyl ester, trans-9-octadecenoic acid methyl ester or a combination thereof.

13. The odor neutralizing composition of claim 5, wherein the one or more fatty acids comprises one or more modifications including analogues, sulfur-substitutions, nitrogen derivatives, methyl additions, methoxy additions, methyl ester additions, hydroxyl additions, carboxyl additions, rings additions or a combination thereof.

14. The odor neutralizing composition of claim 5, wherein the one or more solubilizing agents comprise triethylene glycol, glycols, alkanolamides, silicone glycols, alkyl benzene sulfonates, alcohol ethoxylated, phosphate esters, alkylphenol ether sulfates, alkylaryl sulfonates, nonylphenoxypoly-(ethyleneoxy)ethanol, polyethylene glycol, monostearate, sulfonates, siloxanes, polyglycols, polyvinyl alcohol, polyester glutarate, caprate/caprylate, long chain alkyl ether, dialkyl diester, glutarate, water, ethanol, ethanol, n-propanol, i-propanol, n-butanol, i-butanol, sec-butanol, t-butanol, n-octanol, cyclohexanol, ethyl ether, propyl ether, isopropyl ether, dimethoxyethane, cyclic ether, dioxane, tetrahydrofuran, mono- and di-alkylene glycol monoalkylethers, ethylene glycol monomethylether, ethylene glycol monoethylether, diethylene glycol monoethylether, ketones, acetone, methyl ethyl ketone, methyl isobutyl ketone, cyclohexanone, esters, ethyl acetate, butyl acetate, halogenated hydrocarbons, carbon tetrachloride, chloroform, dichloromethane and 1,2-dichloroethane, aliphatic hydrocarbons, hexane, octane, alicyclic hydrocarbons, cyclohexane, cycloheptane, aromatic hydrocarbons, benzene, toluene, xylene, nitrites, acetonitrile, aprotic polar solvents, N,N-dimethylformamide, dimethyl sulfoxide, pyridine and combinations thereof.

15. The odor neutralizing composition of claim 5, wherein the one or more surfactants comprise tergitol, ethoxylated alkyphenol, alkoxylated linear alcohols, ethoxylated linear alcohols, glycols, polyethylene, mono(p-nonylphenyl)ether, polyoxyethylene alkyl ethers and polyoxyethylenealkylarylether, polyoxyethylenearylether, esters of fatty acids with polyhydric alcohols, nonoxynol-9, polyoxyethylene alkyl ethers, polyoxyalkylene alkyl ethers, polyoxyalkylene alkyl ethers, polyoxyethylene derivatives, xyethylene oxypropylene block copolymers, sorbitan fatty acid esters, polioxyethylene sorbitan fatty acid esters, polioxyethylene sorbitol fatty acid esters, glycerol fatty acid esters, glycerol fatty acid esters, polyoxyethylene fatty acid esters, polyoxyethylene alkyl amines, alkylol amides, fatty acid polyhydric alcohol polyoxyethylenes, fatty acid esters of sucrose, block copolymers of ethylene oxide, propylene oxide, fatty alcohols, fatty esters, glycerol esters, glycol esters, alkyl phenols, acetylenic glycols, alkanolamines, alkanolamides, polyethoxylated mercaptans, sorbitol, sorbitan derivatives, water, methanol, ethanol, n-propanol, i-propanol, n-butanol, i-butanol, sec-butanol, t-butanol, n-octanol, cyclohexanol, ethyl ether, propyl ether, isopropyl ether, dimethoxyethane, cyclic ether, dioxane, tetrahydrofuran, mono- and di-alkylene glycol monoalkylethers, ethylene glycol monomethylether, ethylene glycol monoethylether, diethylene glycol monoethylether, ketones, acetone, methyl ethyl ketone, methyl isobutyl ketone, cyclohexanone, esters, ethyl acetate, butyl acetate, halogenated hydrocarbons, carbon tetrachloride, chloroform, dichloromethane and 1,2-dichloroethane, aliphatic hydrocarbons, hexane, octane, alicyclic hydrocarbons, cyclohexane, cycloheptane, aromatic hydrocarbons, benzene, toluene, xylene, nitrites, acetonitrile, aprotic polar solvents, N,N-dimethylformamide, dimethyl sulfoxide, pyridine and combinations thereof

16. The odor neutralizing composition of claim 5, wherein the one or more surfactants is ethoxylated alkyphenol; the one or more solubilizing agents are triethylene glycol, water and ethanol; and the one or more fatty acids is ricinoleic acid; the one or more organic carboxylic acids is citric acid.

17. A method of neutralizing odors in an enclosure comprising the steps of:
positioning a container comprising an odor neutralizing composition into an enclosure, wherein the odor neutralizing composition consists essentially of one or more surfactants, one or more solubilizing agents, one or more fatty acids and one or more organic carboxylic acids; and
contacting the enclosure with the odor neutralizing composition.

18. The method of claim 17, wherein the container releases the odor neutralizing composition by at least one of spraying, misting, fogging, smoking and atomizing.

19. The method of claim 17, wherein the odor neutralizing composition further comprises one or more propellants.

20. The method of claim 17, wherein the odor neutralizing composition comprises one or more surfactants; one or more solubilizing agents; one or more fatty acids; and one or more organic carboxylic acids.
